(19) United States
(12) Patent Application Publication
(10) Pub. No.: US 2007/0175482 A1
(43) Pub. Date: Aug. 2, 2007

(54) Apparatus for introducing an airway tube into the trachea having visualization capability and methods of use

(75) Inventors: Zebadiah Kimmel, Brookline, MA (US); Raymond Glassenberg, Wilmette, IL (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: EZC Medical LLC, San Francisco, CA (US)

(21) Appl. No.: 11/341,159

(22) Filed: Jan. 27, 2006

(51) Int. Cl. A61B 1/04; A61B 1/06; A62B 9/06

(52) U.S. Cl. 128/207.14; 600/120; 600/160

(57) ABSTRACT

Apparatus for introducing an airway tube within a patient's trachea, and methods of use, are provided, in which the an elongated rigid bougie includes a light source and imaging system configured to be introduced into a patient's trachea using video images received from the imaging system. Once a distal end of the bougie is visually confirmed to be placed with the patient's trachea, an airway tube is advanced over the bougie. The apparatus also may include two separable components, thereby facilitating video-guided placement of one component in pediatric applications under guidance of video images provided by the second component.

### FIELD OF THE INVENTION

The present invention relates to apparatus for introducing an airway tube, such as an endotracheal tube, into a patient's trachea, wherein the apparatus includes visualization capability that assists in placing the airway tube.

### BACKGROUND OF THE INVENTION

In emergency medical management of a patient, it is essential that a patient airway be established in as short of a time as possible. Intubation of the trachea with an airway tube, such as an endotracheal tube, Combi-tube or laryngeal mask airway, is a common form of providing ventilation and administering gaseous medication. Through a properly placed airway tube, air or oxygen can be delivered to the patient in an emergency situation.

Endotracheal tubes and methods for using such apparatus are well known. Unfortunately, not all patients are receptive to endotracheal intubation. For example, in the event that a patient is obese, pregnant, or has laryngeal edema or a short thick neck, routine intubation procedures may become difficult or even risky.

In such situations, the clinician may utilize a bougie. A bougie is essentially a thin elongated member that is inserted into a patient's trachea using a laryngoscope. Once the distal end of the bougie is positioned within the trachea, the laryngoscope is removed and an endotracheal tube is advanced over the proximal end of the bougie and.-into the patient's trachea. At that point, the bougie may be removed and patient ventilation begun. One disadvantage with this method of intubation is the necessity of manually viewing the laryngeal opening using a laryngoscope, a task that is often difficult—for example, when the patient is obese, or is a child. Accordingly, it would be desirable to provide an airway introducer apparatus that does not require the use of a laryngoscope.

Additionally, another problem with the previously-known bougies is the lack of confirmation that the endotracheal tube was positioned correctly. If the endotracheal tube is mistakenly placed in the patient's esophagus, subsequent ventilation may be ineffective, leading to asphyxiation. Accordingly, it would be desirable to provide an airway introducer apparatus that enables the clinician to confirm proper placement of an airway tube within the trachea.

Recent attempts at refining the intubation procedure have focused on providing a stylet or scope that is equipped with a camera and light source, thereby avoiding the use of a laryngoscope. For example, U.S. Pat. No. 6,115,523 to Choi, et al., describes an imaging scope comprising a sheath that houses a plastic fiberoptic bundle and a malleable stylet, which is inserted through the lumen of a conventional endotracheal tube. The combined device may be bent to a desired curvature prior to insertion within a patient.

One drawback of the Choi device is the relatively large size of the components that must be inserted into the patient. Generally, the larger the components that must be inserted into the patient, the more difficult that intubation becomes. Accordingly, it would be desirable to provide apparatus for introducing an airway tube that is smaller than previously-known stylets or scopes.

In addition, the malleable stylet used in the Choi device may become reshaped during insertion of the device through the patient's oropharyngeal area, thereby resulting in improper placement of the distal end of the endotracheal tube. It would therefore be desirable to provide apparatus for intubating a patient that is not prone to reshaping during rigorous insertion efforts.

Additionally, intubation of pediatric patients is often difficult due to the small anatomical features of such patients. Many previously-known visualization devices are simply too large to be effective in pediatric applications. Health care workers thus must perform such intubations using previously-known laryngoscopes to manually view the relevant anatomical landmarks, which is a difficult task and prone to failure. It therefore would be desirable to provide an airway introducer apparatus that may be used on pediatric patients without the need for a laryngoscope.

### SUMMARY OF THE INVENTION

In view of the above-listed disadvantages of the prior art, it is an object of the present invention to provide apparatus for introducing an airway tube into a patient's trachea, and methods of use, that avoid the use of previously-known laryngoscopes.

It is a further object of the present invention to provide apparatus for introducing an airway tube into a patient's trachea, and methods of use, that permit a health worker to confirm the proper placement of an airway tube.

It is another object of this invention to provide apparatus for intubating a patient that includes a preformed curvature that is not prone to reshaping during rigorous insertion efforts.

It is yet a further object of the present invention to provide apparatus for introducing an airway tube into a patient's trachea, and methods of use, suitable for use in pediatric applications, and without the need for a laryngoscope.

These and other advantages may be accomplished by providing apparatus for introducing an airway tube into a patient's trachea, and methods of use, wherein the apparatus includes a video sensor for visualizing the laryngeal inlet and thus obviates use of a laryngoscope. In some embodiments, the camera is configured for translation on the apparatus, thereby enabling its separate use with pediatric patients.

The apparatus of the present invention comprises a thin stiff elongated body having a video sensor and illumination apparatus associated therewith. The elongated body preferably has a defined curvature relative to its longitudinal axis and may optionally include a bend near its distal end. In accordance with the principles of the present invention, the video sensor preferably comprises a complementary metal oxide semiconductor (“CMOS”) circuit having a small frontal profile, thereby providing a reduced insertion diameter.

The pixel array of the video sensor may have any of a variety of configurations, although preferably the driver circuitry for the pixel array is disposed substantially perpendicular to the plane of the pixel array, or is located remote from the pixel array. Preferably, the video sensor is configured so that its output may be connected directly to a viewing device, such as a monitor or television, without intermediate signal processing. In this manner, the health worker may observe the airway introduction process in real-time.

The apparatus also may include a protrusion disposed near the distal end of the device, distal to the video sensor. This arrangement enables the user to gain some perspective of the interior of the patient's trachea, facilitates guiding the present inventive device into the trachea (because the protrusion is small and therefore fits more easily within the narrow space of the laryngeal inlet), and confirms proper placement of the apparatus. The protrusion may be fixed or articulable.

In accordance with another aspect of the present invention, the apparatus comprises two sections configured to be translated relative to one another, or even separated. This feature is expected to be especially advantageous for pediatric patients and others presenting difficult intubation scenarios. In particular, this configuration allows a portion of the apparatus to be inserted into a pediatric patient's trachea under guidance of the visualization device. Once positioned, the section of the airway introduction apparatus containing the visualization equipment may be separately removed, while the other portion of the apparatus remains in the patient's trachea to provide a guideway for introduction of an endotracheal tube or other airway tube.

Methods of using the apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIGS. 1A and 1B are perspective and schematic views, respectively, of an airway introducer apparatus of the present invention;

FIG. 2 is a perspective view of an alternative embodiment of an airway introducer apparatus of the present invention;

FIGS. 3A-3D are, respectively, schematic views of alternative embodiments of proximal and distal end arrangements suitable for use in the airway introducer apparatus of FIG. 2;

FIGS. 4A-4C are perspective views illustrating possible configurations of using a further alternative embodiment of the apparatus of the present invention;

FIGS. 5A-5B are sectional views of the apparatus of FIGS. 4A-4C; and

FIGS. 6A-6C depict methods of using the apparatus of FIG. 3 to intubate a patient.

### DETAILED DESCRIPTION OF THE INVENTION

The present invention is directed to apparatus for introducing an airway tube, such as an endotracheal tube, Combi-tube or laryngeal mask airway, into a patient's trachea, wherein the apparatus includes a video sensor that enables the health worker to visualize the intubation process and confirm proper placement of the airway tube. The apparatus of the present invention advantageously provides a relatively stiff curved bougie and a pixel array with reduced frontal profile that enables the apparatus to be used in pediatric applications and other difficult intubation scenarios without the use of a laryngoscope.

In accordance with one aspect of the invention, the apparatus includes a protrusion disposed at or near the distal end, distal of the video sensor. The protrusion may be fixed, translatable relative to the video sensor, articulable or removable and assists in completing the intubation process. Optionally, the protrusion may comprise a portion of a separable bougie, thereby enabling the apparatus to be separated into at least two components during the intubation process. In this way, the video sensor may be used to confirm entry of the protrusion into the patient's laryngeal inlet. The video sensor portion of the apparatus is withdrawn, while leaving the bougie in position to serve as a guideway over which the airway tube then may be advanced.

Advantageously, the video sensor of the present invention is configured to provide an output that may be directly coupled to a monitor or other viewing device, without intermediate signal processing. When a health worker inserts the apparatus into a patient's trachea, advancement of the apparatus may thus be monitored so that the distal tip is directed into the patient's laryngeal inlet. Once the distal tip of the apparatus is placed in the patient's trachea, the apparatus may be disconnected from the monitor and an airway tube, e.g., endotracheal tube, advanced over the apparatus. Next, the health worker then may reconnect the video sensor to the monitor and confirm proper placement of the airway prior to removing the apparatus and initiating ventilation.

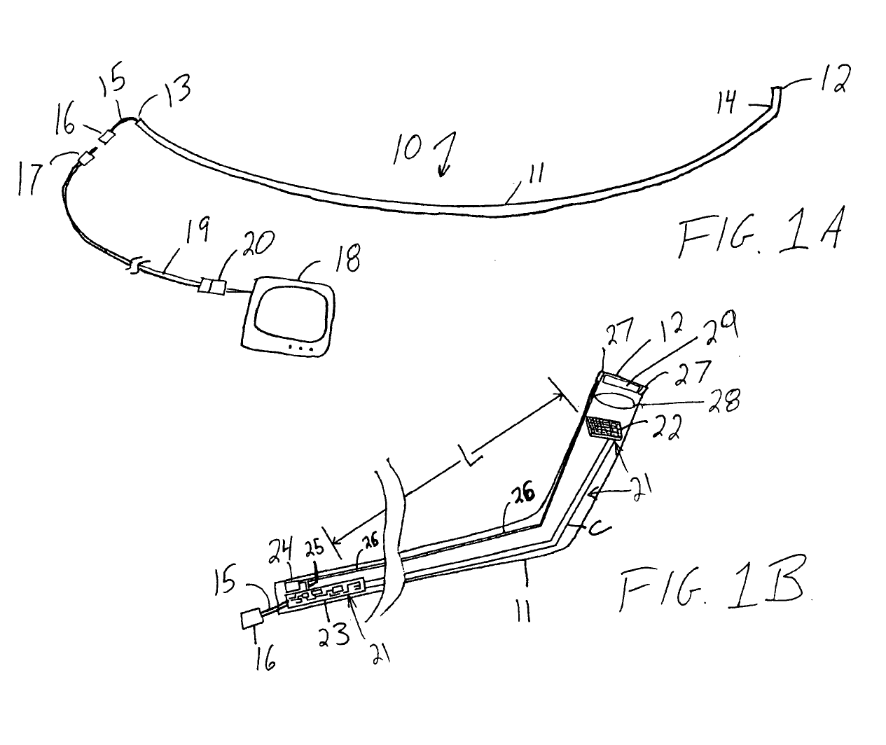

Referring to FIGS. 1, an embodiment of an airway introducer apparatus constructed in accordance with the principles of the present invention is described. Apparatus 10 comprises elongated body 11 having distal end 12 and proximal end 13. Elongated body 11 is curved or arcuate, and optionally includes bend 14 disposed near distal end 12. Elongated body 11 preferably is formed from a relatively rigid material, such as a hard polymer, metal or metal alloy, and ensures that apparatus 10 retains its shape when subjected to the loads expected to be encountered while inserting apparatus 10 orally into a patient's trachea. Apparatus 10 alternatively may retain some degree of flexibility, which allows the pre-formed curvature to vary during insertion. In addition, bend 14 preferably is sufficiently flexible to permit passage of an airway tube over the bend.

Apparatus 30 preferably is about 40 cm long between distal end 12 and proximal end 13. Elongated body 11 may have an essentially circular cross section with a diameter of approximately 4 to 8 mm. In other embodiments, elongated body 11 may have a non-circular cross section. For example, an oval shape may prove provide enhanced flexibility in one direction, while reduced flexibility in an orthogonal direction. Apparatus 10 should have a sufficiently narrow profile that it may pass through the lumen of an conventional airway tube, such as an endotracheal tube.

Apparatus 10 further comprises conduit 15 having connector 16 for transferring data to a video monitor. Connector 16 may be selectively coupled to connector 17 to communicate with monitor 18 via conduit 19 having optional connectors 20. Monitor 18 may be a previously known video screen, television, or other known image display device. Preferably, the video signal output by apparatus 10 may be communicated directly to monitor 18 for real time observation of the insertion process, and without intermediate signal processing.

Conduit 15 preferably comprises a wire, cable, or other medium for transmitting electrical signals, whereas connector 16 or 17 may be an RCA jack, RCA plug, or similar apparatus that preferably allows rapid connection. It will be understood that the present invention is not limited to wired data transfer, but alternatively, the output signal may be provided to monitor 18 wirelessly using radio waves, infrared, microwaves, or other medium.

With respect to FIG. 1B, apparatus 10 includes imaging system 21 disposed within an interior space of apparatus 10 near distal end 12. Imaging system 21 preferably comprises a CMOS chip, and more preferably comprises a CMOS chip with analog output that can directly interface with video hardware, e.g., using a NTSC/PAL format. CMOS chips having direct analog output capability, e.g., using NTSC/PAL format, are commercially available, such as models OV7940 and OV7941 available through OmniVision Technologies, Inc., of Sunnyvale, Calif. Advantageously, outputting a direct analog signal obviates the need for intermediate circuitry to convert the digital image signals into analog image signals. In other embodiments, a chip of standard configuration may be utilized.

In a preferred embodiment, imaging system 21 has a focal length of approximately 4 to 5 cm. Alternatively, imaging system 21 may have focusing capabilities, such as through use of lenses. Imaging system 21 may have a field of vision that is as wide as possible, preferably at least 70 degrees and more preferably 100 to 120 degrees.

In accordance with one aspect of the present invention, imaging system 21 has a reduced frontal profile that provides a reduced insertion profile for apparatus 10. In previously-known imaging systems, the driver circuitry for the video sensor is disposed adjacent to, and in the same plane as, the pixel array. In the video sensor of the present embodiment, however, pixel array 22 is disposed remote from array driver circuitry 23 and is in electrical communication with that device. Array driver circuitry 23 may include capacitors or other electronic components and may be disposed on a relatively rigid circuit board that extends in an asymmetric, elongated manner proximal to pixel array 22. More preferably, however, array driver circuitry 23 is disposed on a printed circuit board formed on a flexible polymeric material. Even more preferably, array driver circuitry 23 may be located some distance L from pixel array 22 and remains in communication with that component via appropriate conduits C. This arrangement may reduce the profile of the distal camera portion of the apparatus and allow the apparatus to more readily pass through the curvature of a surrounding airway tube. For example, distance L may be selected such that array driver circuitry 23 is located at or near proximal end 13, as shown in FIG. 1A.

Circuitry 23 preferably provides analog output readable by hardware using NTSC/PAL technology. In this manner, circuitry 23 may omit analog-to-digital converter circuitry and thereby reduce the number of required components. Imaging system 21 further may be reduced in size by omitting the infrared filter commonly employed with CMOS chips. Circuitry 23 optionally may further comprise a timer that inactivates one or more features of apparatus 10 after a pre-specified amount of time has elapsed, thereby preventing apparatus 10 from being used or re-used indefinitely.

In accordance with another aspect of the present invention, the imaging system may comprise a pixel array that is substantially tangential to the longitudinal axis of the apparatus. In this configuration, the pixel array may be used to gather images lateral to the apparatus, or may be used in conjunction with a mirror, prism, or other optical device that redirects light rays arriving from a distal location so that the light rays are redirected onto the pixel array.

Video data is transmitted from imaging system 21 via conduit 15. Imaging system 21 receives power from power source 24, which is transmitted via conduit 25. Power source 24 may comprise an internal source of power, such as one or more “AAA” size batteries. Alternatively, power source 24 may comprise an external source of power, such as an external battery or an A/C socket with appropriate converters and/or connectors. Preferably, power source 24 is selectively actuatable, such as by a switch, to prevent loss of power when apparatus 10 is not in use.

Apparatus 10 further comprises light source 27 disposed adjacent to or surrounding pixel array 22. Light source 27 comprises one or more LEDs or other illumination sources. Preferably, light source 27 is configured as an annulus disposed near distal end 12 and directs light in a distal direction, as illustrated in FIGS. 1. Power may be transmitted to light source 27 via conduit 26, either directly from power source 24 or by connection to another powered component, such as imaging system 21.

Lens 28 preferably is disposed between imaging apparatus 21 and distal end 12, and may be formed from glass, polymer, liquids such as oil and water, or other optically suitable substance. Lens 28 may comprise a simple convex lens, or may comprise a prism or other component for redirecting light rays to accommodate alternate configurations of imaging apparatus 21.

Optional shield 29 also may be disposed between lens 28 and exterior of apparatus 10. Shield 29 prevents moisture, bodily fluids, and other debris from contacting lens 28. In other embodiments, lens 28 is at least partially exposed to the surrounding environment, and may further comprise a hydrophobic or hydrophilic coating. A hydrophobic or hydrophilic coating also may be applied to optional shield 29.

Apparatus 10 provides significant advantages compared to previously-known bougies. As described, for example, in an article entitled Difficult Airway Society guidelines for management of the unanticipated difficult intubation, Anaesthesia, July; 59(7): 675-94 (2004), a bougie is a flexible and malleable apparatus that may be used to feel for the patient's trachea. The bougie is often used with a Macintosh laryngoscope during difficult intubations.

In use, the anesthesiologist places the laryngoscope in a patient's mouth while the bougie is blindly inserted into the patient's oral cavity. The anesthesiologist then manipulates the bougie while attempting to detect tactile “clicks” that indicate placement of the bougie in the trachea. The article notes that even though proper placement may occur, there may be an absence of clicks if the bougie is in the center of the tracheal lumen. Likewise, no clicks are felt if the bougie is improperly placed in the esophagus. The disadvantages of the bougie become even more apparent when one considers the difficulties that may arise outside a hospital setting, such as for a paramedic trying to feel for clicks in the back of a moving ambulance in an emergency situation.

By contrast, apparatus 10 of the present invention may be successfully employed by those having significantly less training or experience than anesthesiologists. Apparatus 10 may be activated and coupled to viewing screen 18. Upon activation, light source 27 illuminates and imaging system 21 begins to transmit signals. The health worker then inserts apparatus 10 into the patient's mouth and observes the path of insertion by watching monitor 18. The curvature of apparatus 10 and bend 14 are pre-formed to assist in directing distal tip 12 towards the trachea. Accordingly, the user will be able to observe the glottic opening displayed on monitor 18, and maneuver the apparatus 10 until its distal end passes through the laryngeal inlet and into the trachea.

Unlike previously-known bougies, apparatus 10 permits proper placement to be confirmed even when positioned in the midline of the tracheal lumen. Apparatus 10 may be momentarily disconnected to allow the endotracheal tube to be inserted around apparatus 10 and into the patient's trachea. Alternatively, apparatus 10 may be “preloaded” through the endotracheal tube, such that the endotracheal tube is positioned around conduit 15 or 19 as apparatus 10 is inserted into the patient. Moreover, apparatus 10 provides positive confirmation of proper placement in the trachea by displaying an image of the distal end of the endotracheal tube following insertion of the endotracheal tube over apparatus 10 and into the patient. In the event that the airway tube is improperly positioned, it may be repositioned under the guidance as need using the images provided by apparatus 10.

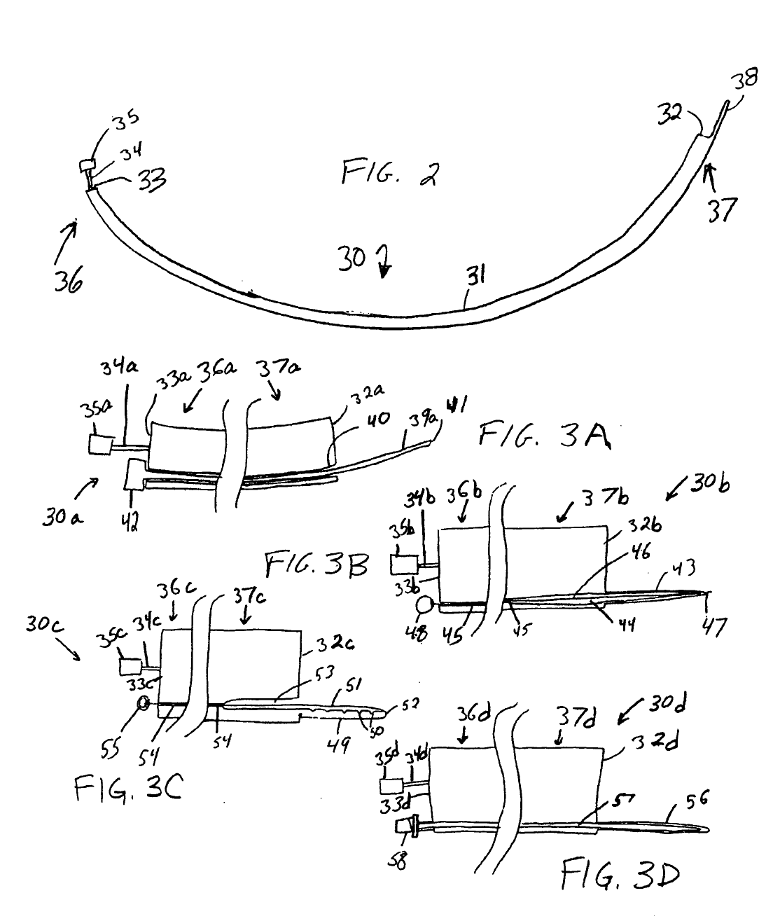

Referring now to FIGS. 2 and 3, an alternative embodiment of the airway introducer apparatus of the present invention is described. FIG. 2 provides a schematic view of apparatus 30, while FIGS. 3A-3D provide alternative arrangements for the proximal and distal ends of the apparatus of FIG. 2.

Apparatus 30 comprises elongated body 31 having distal end 32 and proximal end 33, and is similar in design and construction to apparatus 10, discussed above. Elongated body 31 preferably has a preformed curvature, and optionally may include a bend near distal end 32. Conduit 35 terminates in connector 36 which enables the apparatus to be selectively connected to monitor 18 using suitable connectors and conduits, as described herein above. Apparatus 30 further comprises an imaging system and a light source, which components are coupled to a power source via appropriate conduits and connectors. In embodiments comprising an external power source, electrical conduits and connectors preferably extend from proximal region 36.

In accordance with one aspect of the present invention, apparatus 30 includes protrusion 38 disposed in distal region 37, which extends beyond distal end 32. Preferably, protrusion 38 extends approximately 1 to 2 cm beyond distal tip 32, and comprises a stiff extension member that is at least partially within the optical field of the imaging system. Protrusion 38 preferably has a blunt atraumatic tip to avoid injury to the interior wall of the patient's trachea. Alternatively, the tip of protrusion 38 may be shaped in such a way as to facilitate entrance into the glottic opening, for example, it may end in a bulb or sphere.

In FIG. 2, protrusion 38 is fixed to the inferior aspect of distal end 37 of apparatus 30, although other embodiments may position the protrusion at other suitable locations, preferably at or near the distal end. In accordance with one aspect of the present invention, the progress of protrusion 38 may be observed on monitor 18 while apparatus 30 is inserted into the patient's trachea. Because the profile of protrusion 38 is smaller than that of elongated body 31, a user may find it easier to guide protrusion 38 into the laryngeal inlet, as compared to an embodiment of the apparatus having a blunt distal region without protrusion 38. Once protrusion 38 has been successfully inserted into the trachea, apparatus 30 may be advanced, to follow the path of protrusion 38 and enter the trachea. This procedure may be facilitated by manually rotating apparatus 30 during advancement.

Alternatively, it may be advantageous for the protrusion to be translatable or even removable. For example, once the distal end of the elongated member has entered the trachea, the protrusion may no longer be required and may be translated proximally or removed entirely. Alternatively, if the camera has a particularly advantageous view of the glottic opening, it may be desirable to advance the protrusion forward while keeping the camera stationary. In the following description, components of FIGS. 3A-3D similar to those of FIG. 2 are indicated by like reference numerals including a letter suffix. Thus, for example, in the embodiment of FIG. 3A, the elongated body is indicated as “31a”.

FIG. 3A depicts proximal region 36a and distal region 37a of an alternative embodiment of apparatus 30 of FIG. 2, wherein protrusion 39 comprises an elongated member slidably disposed within lumen 40 that passes through elongated body 31a. Protrusion 39 has atraumatic distal tip 41 to prevent trauma to the patient, and enlarged proximal end 42. End 42 prevents the user from inserting protrusion 39 too far within lumen 40, and facilitates manipulation of the protrusion by the user. In this regard, protrusion 39 may be advanced, retracted, and rotated, which may assist insertion. Protrusion 39 is configured such that distal tip 41 extends approximately 2 cm beyond distal end 32a when proximal end 42 is in contact with proximal end 33a.

In use, the clinician may advance the apparatus 30a, as described above for apparatus 30, until a location is reached in which protrusion 39 is disposed within the patient's trachea. Apparatus 30a then is advanced slightly further, such that distal tip 32a is within the trachea. Protrusion 39 then may be withdrawn proximally using enlarged proximal end 42 until distal tip 41 is retracted into lumen 40. The remainder of the intubation procedure then may be continued as discussed above.

Referring now to FIG. 3B, another alternative embodiment of is described, in which protrusion 43 comprises a thin hollow member affixed near distal end 32b. Protrusion 43 is adjacent to space 44, which narrows into lumen 45. Lumen 45 extends from space 44, 30 through apparatus 30b and to proximal end 33b. Wire 46 passes through lumen 45, and has a distal end affixed to the interior of distal tip 47 of protrusion 43. The proximal end of wire 46 is coupled to handle 48, which preferably is located some distance outside of lumen 45.

Use of this embodiment is similar to that described for the embodiment of FIG. 3A above. Once protrusion 43 is disposed in the trachea, and distal end 32b is advanced into the trachea; protrusion 43 then may be retracted into space 44 by the clinician by applying a proximally-directed force on handle 48. Once protrusion 43 is retracted, the airway tube may be inserted as described above.

Referring now to FIG. 3C, another embodiment of a retractable protruding member is described. Protrusion 49 is attached to distal end 32c of apparatus 30c, and comprises a flexible rod having a plurality of cutouts or notches 50. Notches 50 provide weak points that allow articulation of protrusion 49 to occur at pre-selected locations. Wire 51 is affixed to distal tip 52 of protrusion 49 and passes through space 53 and lumen 54, terminating at handle 55. Space 53 and lumen 54 are similar to space 44 and lumen 45 of the embodiment of FIG. 3B. Handle 55 is selected such that it is larger than lumen 54. When a proximally-directed force is applied to handle 55, wire 51 applies force to distal tip 52, and protrusion 49 articulates at notches 50. As protrusion 49 articulates, the force transmitted along wire 51 pulls distal tip 52 and connected segments of protrusion 49 into space 53. It should be understood that space 53 is sized to accommodate retraction of protrusion 49.

As for the preceding embodiment, apparatus 30c is inserted such that protrusion 49 and distal end 32c are disposed within the patient's trachea. The user may then apply proximally-directed force to handle 55, causing protrusion 49 to articulate into space 53. The remainder of the intubation procedure may continue as described above.

Referring now to FIG. 3D, a further alternative embodiment of a retractable protruding member is described. In this embodiment, protrusion 56 comprises a hollow member that extends distally from distal end 32d and is selectively inflatable and deflatable. Lumen 57 passes through apparatus 30d and provides fluid communication between protrusion 56 and fitting 58. Lumen 57 may extend beyond proximal end 33d, and as such may include a tubular section, such as a flexible polymer tube. Fitting 58 may be a Luer-lock fitting, a simple pressure release valve, or other appropriate fitting.

Protrusion 56 preferably is inflated with air or other gas or fluid via fitting 58 to provide a relatively stiff member. Fitting 58 may include a valve or other apparatus to prevent fluid from being released inadvertently. When desired, fitting 58 may be actuated to release fluid and cause protrusion 56 to become limp. Actuation of fitting 58 may involve opening a valve, removing a plug, providing suction using a syringe, or other manner of releasing fluid. In some embodiments, fluid may be reinserted into lumen 57 through fitting 58, such as if fitting 58 comprises a Luer-lock fitting and fluid is being inserted with a syringe.

In use, apparatus 30d is inserted into the patient with protrusion 56 in the inflated configuration. Once protrusion 56 and distal end 32d are disposed in the patient's trachea, fluid is released through fitting 58, causing protrusion 56 to become limp. The remainder of the intubation procedure may be carried out as described above.

It should be noted that for each of the above-described embodiments, the imaging system may be used to confirm placement of the airway tube. Once the airway tube has been placed, the apparatus may be used to visually confirm that the distal end of the airway tube is disposed within the trachea. The presence of visual indicia on the elongated member, e.g., a series of longitudinally spaced-apart rings or other markings, also may be used to confirm proper placement of the distal end of the apparatus in the trachea. After observing the distribution of such visual indicia on the monitor, the apparatus may be removed and ventilation initiated.

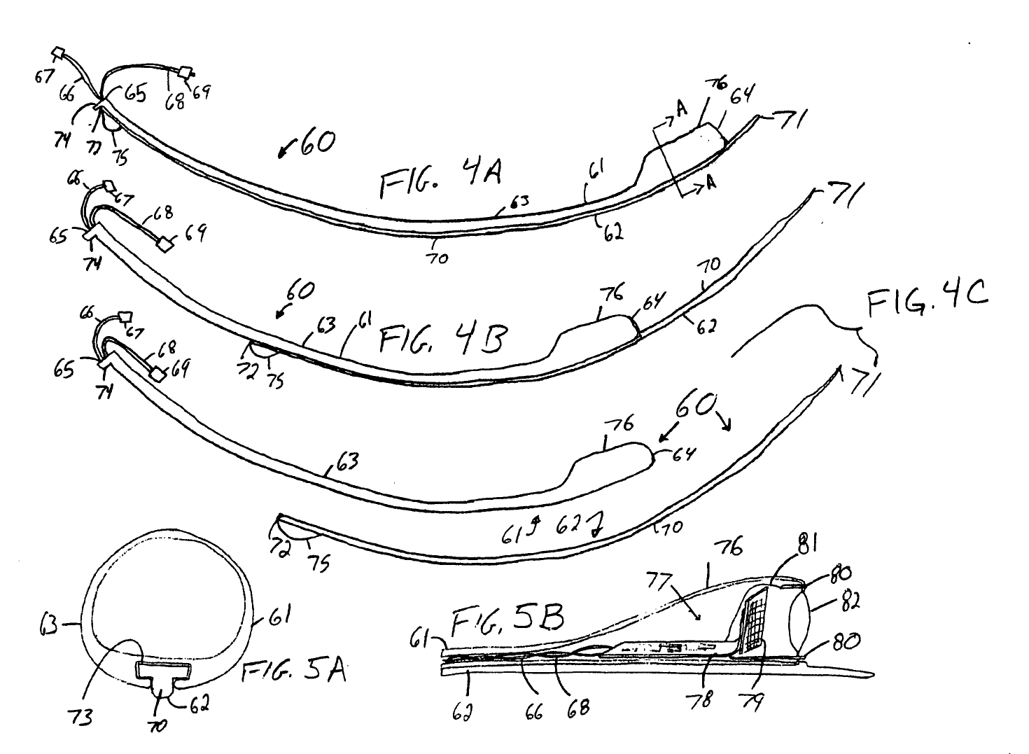

Referring now to FIGS. 4, another alternative embodiment of the apparatus of the present invention is described. Apparatus 60 is especially advantageous for pediatric patients and others having smaller anatomical features, but may be used on a wide variety of patients. Apparatus 60 comprises main section 61 and introducer section 62, which may be selectively coupled.

Main section 61 preferably comprises elongated body 63 having distal end 64 and proximal end 65. Section 61 need not be as stiff as in preceding embodiments, and preferably follows the shape of introducer section 62, described in greater detail below. Main section 61 further comprises the features of apparatus 10 described above, including an imaging system, light source, and pre-formed curvature. Power source is located external to apparatus 60, so in this embodiment power is transmitted to the imaging system and light source via conduit 66 and connector 67. For example, power source may be an A/C wall switch with appropriate conduits, connectors, and converters to connect and provide the appropriate direct current to apparatus 60. Alternatively, apparatus 60 may comprise an internal power source that preferably is disposed near proximal end 65. Video signals output from the imaging system are transmitted via conduit 68 and connector 69 to monitor 18. It should be understood that here, as in other embodiments, the power conduit and the video conduit optionally may be contained in a single main conduit.

Introducer section 62 comprises elongated body 70 having a predefined curvature, distal end 71 and proximal end 72. Distal end 71 preferably is atraumatic (e.g., blunt or rounded) to prevent trauma to the patient. Introducer section 62 preferably has a diameter of 1 to 4 mm, enabling it to fit within a lumen of a pediatric airway tube. Of course, larger diameters may be used for adult airway tubes. Preferably, introducer section 62 is formed of a stiff material, such as a relatively rigid plastic, metal, or metal alloy, and is about 1 to 2 cm longer than main section 61, such that distal end 71 may protrude beyond distal end 64. Proximal end 72 of introducer section 62 may be flush with proximal end 65 of main section 61, or alternately may be several centimeters longer, thereby providing a graspable handle by which the operator may manipulate the translation of distal end 71 relative to main section 61 if desired.

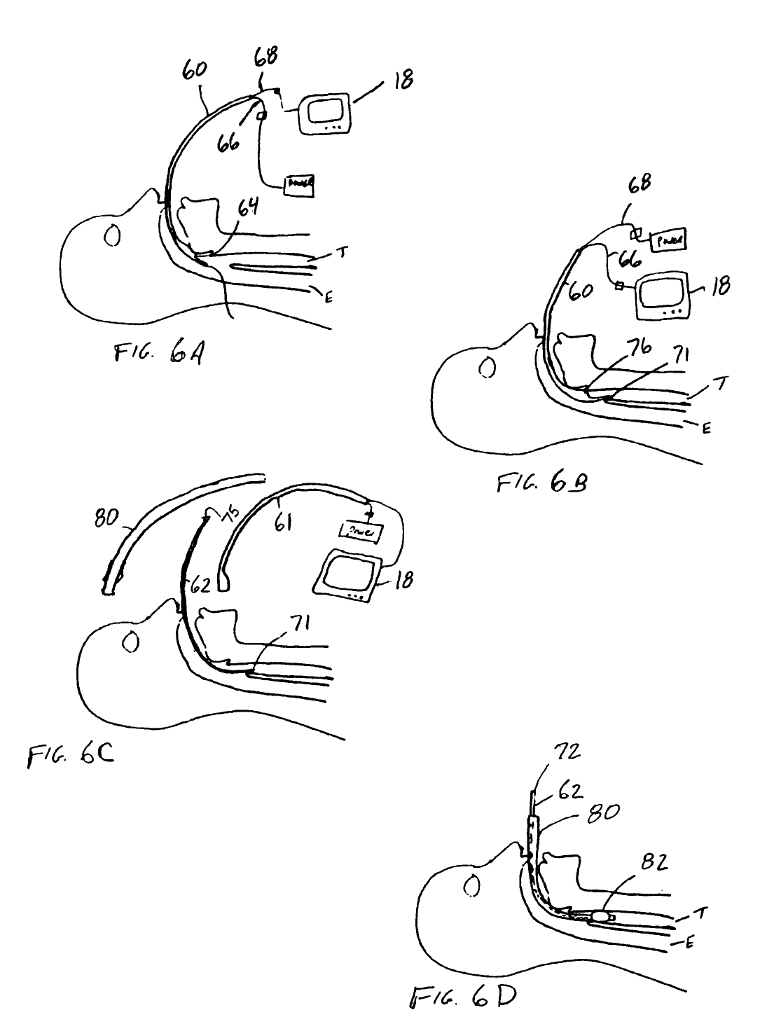

In accordance with one aspect of the present invention, main section 61 and introducer section 62 are slidably engagable, thereby allowing main section 61 to translate along the length of introducer section 62, while following the shape of the introducer section. One such arrangement is depicted in FIG. 5A (for clarity internal components have been omitted from FIG. 5A). In FIG. 5A, channel 73 is disposed along elongated body 63 of main section 61. A portion of elongated body 70 of introducer section 62 is keyed to mate with channel 73, such that main section 61 may slide along introducer section 62 without becoming disengaged.

Referring again to FIG. 4A, apparatus 60 is depicted with main section 61 fully engaged with introducer section 62. In this position, flange 74 near proximal end 65 of main section 61 contacts proximal end 72 of introducer section 62, thereby preventing the main section from further advancement along the introducer section. Flange 74 need not protrude from proximal end 65, as simply blocking channel 73 also would limit travel of main section 61 in the distal direction. Likewise, the same result may be accomplished by altering the profile of introducer section 62 near distal end 71, such as by adding a widened portion that is too large to fit within channel 73.

Referring now to FIG. 4B, apparatus 60 is depicted with main section 61 translated proximally relative to introducer section 62. Main section 61 and introducer section 62 thus are engaged over a shorter length than in FIG. 4A, but remain securely fastened to one another via the engagement feature described with respect to FIG. 5A.

Relative movement between main section 61 and introducer section 62 is facilitated by grip 75 on introducer section 62. Grip 75 preferably is located along the underside of introducer section 62 and allows a user to securely maintain the position of introducer section 62 as main section 61 is retracted proximally. In accordance with one aspect of the present invention, grip 75 is mechanically coupled to introducer section 62 with quick disconnect tabs or other releasing feature that allows grip 75 to be quickly removed. Alternatively, grip 75 may be coupled to introducer section 62 using a hinge and may be folded down against introducer section 62 to reduce the profile of the introducer section. In accordance with one aspect of the present invention, grip 75 comprises a textured surface of introducer section 62. In another aspect of the present invention, main section 61 contains fenestration or apertures along its sides, by which the operator may grasp introducer section 62.

In FIG. 4C, main section 61 has been withdrawn sufficiently in a proximal direction to disengage from introducer section 62, thereby permitting components 61 and 62 to be separately manipulated.

Main section 61 of the embodiment of FIGS. 4 lacks a uniform profile, and instead includes enlarged region 76 disposed near distal end 64. Enlarged region 76 and the non-uniform profile of main section 61 permits the overall insertion profile of the device to be reduced, thereby facilitation insertion of apparatus 60 in a patient that has small anatomical features.

As depicted in FIG. 5B, enlarged region 76 houses imaging system 77, including array driver circuitry 78 and pixel array 79. Pixel array 79 preferably is disposed substantially perpendicular to circuitry 78 to reduce the profile of apparatus 60, but other configurations may be employed. For example, light received by the lens of the imaging system may be diverted using a prism or a mirror to direct the light onto an array that is substantially coplanar with the circuitry of the imaging system. Imaging system 77 communicates with a suitable power source and monitor 18 via conduits 66 and 68, respectively. Even more preferably, array driver circuitry 78 may be located some distance L from pixel array 79 and remain in communication with that component via appropriate conduits. This arrangement may reduce the profile of the distal camera portion of the apparatus. For example, distance L may be selected such that array driver circuitry 78 is located at proximal end 65.

Light source 80 is in electrical communication with the power source via conduit 81, which may be the same as conduit 66 or via a connection to imaging system 77 or other component. Light source 80 preferably is an LED configured to form an annulus surrounding lens 82. Light source 80 also may comprise one or more LEDs configured in other positions.

Lens 82 is optically disposed between imaging system 77 and the exterior of main section 63. Lens 82 and imaging system 77 preferably are to image an area distal to main section 61 and including distal end 71 of introducer section 62 when apparatus 60 is positioned as depicted in FIG. 4A. Optionally, lens 82 and light source 80 may be shielded by a transparent faceplate (not shown).

Referring now to FIGS. 6, methods of using apparatus 60 are described, for example, to intubate a pediatric patient. Apparatus 60 is attached to a power source and monitor 18 via conduits 66 and 68, respectively. Once so connected, images are transmitted from imaging system 77 to monitor 18, where they may be observed by the clinician or health worker. As depicted in FIG. 6A, apparatus 60 is inserted orally into the patient, and advanced through the oropharyngeal area until the distal end of introducer section 62 approaches the epiglottis.

The clinician continues advancing apparatus 60, while observing the patient's larynx on monitor 18. The clinician then manipulates apparatus 60 so that distal end 71 passes through the larynx and into trachea T, as depicted in FIG. 6B. Proper placement of distal end 71 in trachea T may be confirmed by observing the presence of visual indicia, e.g., tracheal rings, near distal end 71 on monitor 18. The absence of such rings is an indication that distal end 71 has been improperly directed into esophagus E, and that apparatus 60 should be repositioned. Notably, the relatively smaller anatomy of a pediatric patient is expected to prevent enlarged portion 76 from entering trachea T.

Once the clinician confirms proper placement of distal end 71 in trachea T, introducer section 62 is held stationary while a proximal force is applied to retract main section 61 proximally. This proximal force causes main portion 61 to translate proximally away from distal end 71 until components 61 and 62 separate, as depicted in FIG. 6C. Main portion 61 then is put aside and a suitable airway tube 80, illustratively an endotracheal tube, is obtained.

Airway tube 80 is orally inserted into the patient such that introducer section 62 is disposed within the lumen of the airway tube. The clinician continues advancing the airway tube over introducer section 62 and into trachea T, with the clinician rotating the airway tube as needed until it passes through the larynx and into trachea T. During at least a portion of this process, grip 75 may be used to keep introducer section 62 stationary within the trachea, and optional markings (such as ring markings spaced at one-centimeter intervals) on the exterior of introducer section 62 may be used to confirm that introducer section 62 has been kept stationary. Once the airway tube is properly positioned in the trachea, the cuff of the airway tube is inflated to secure the airway tube in position, as shown in FIG. 6D. Introducer section 62 then may be removed from the lumen of the airway tube and ventilation initiated.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. Apparatus for introducing an airway tube into a patient's trachea, the apparatus comprising:
a rigid elongated body having a proximal end, a distal end, and an interior space disposed near the distal end;
an imaging system disposed within the interior space, the imaging system electrically coupled to a connector disposed at the proximal end of the elongated body, the connector configured to be coupled to a monitor;
a light source disposed within the interior space adjacent to the imaging system; and
a optical lens associated with the imaging system to focus light rays onto the imaging system,
wherein the elongated body has a preformed curvature between its proximal and distal ends and is dimensioned to be received within a lumen of the airway tube.

2. The apparatus of claim 1 further comprising a protrusion extending from the distal end of the elongated body.

3. The apparatus of claim 2 wherein the protrusion is fixed to the distal end of the elongated body.

4. The apparatus of claim 2 wherein the protrusion is movable relative to the distal end of the elongated body.

5. The apparatus of claim 4 wherein the protrusion comprises an elongated member configured to reciprocate through a lumen in the elongated body.

6. The apparatus of claim 4 wherein the protrusion comprises a tension member having a first end coupled to the protrusion and a second end coupled to a handle.

7. The apparatus of claim 6 wherein the protrusion comprises a hollow member that is configured to be at least partially retracted within a space in the elongated body.

8. The apparatus of claim 6 wherein the protrusion is configured to articulate to be retracted within a space in the elongated body.

9. The apparatus of claim 4 wherein the protrusion comprises an inflatable member.

10. The apparatus of claim 2 wherein the imaging system comprises a CMOS device.

11. The apparatus of claim 10 wherein the CMOS device comprises a pixel array and driver circuitry, the pixel array configured substantially perpendicular to the driver circuitry.

12. The apparatus of claim 10 wherein the CMOS device comprises a pixel array and driver circuitry, the pixel array disposed at a distance proximal to the array driver circuitry and in communication with driver circuitry.

13. The apparatus of claim 1 wherein the elongated body includes a bend near the distal end.

14. The apparatus of claim 1 further comprising a circuit, the circuit having a timer that disables one or more operative features of the apparatus after a pre-determined time.

15. Apparatus for introducing an airway tube within a patient's trachea, the apparatus comprising:
a main section having an elongated body, a proximal end, a distal end, an imaging system, and a light source; and
an introducer section comprising an elongated body having a proximal end, a distal end, and an engagement feature configured to slidably couple with the main section, the introducer section configured to be received within a lumen of the airway tube.

16. The apparatus of claim 15 wherein the introducer section is more rigid than the main section.

17. The apparatus of claim 16 wherein the imaging system is configured to be selectively directly coupled to a monitor.

18. The apparatus of claim 17 wherein the main section has a non-uniform profile.

19. The apparatus of claim 18 wherein the main section is configured to be selectively coupled to an external power source.

20. A method of introducing an airway tube into a patient's trachea, the method comprising:
providing apparatus comprising an elongated body having a proximal end, a distal end, an imaging system, and a light source, the imaging system configured to communicate video data to an external monitor;
providing an airway tube;
inserting the apparatus orally into a patient;
observing data communicated from the imaging system to determine the position of the distal end of the apparatus relative to the patient's trachea;
advancing the airway tube along the apparatus so that a distal end of the airway tube enters the patient's trachea; and
removing the apparatus while leaving the airway tube in position.

21. The method of claim 20 further comprising observing video images generated by the imaging system to confirm placement of the airway tube.

22. The method of claim 21 wherein the apparatus comprises two components, the method further comprising:
after observing data communicated from the imaging system to determine the position of the distal end of the apparatus relative to the patient's trachea, removing a first component of the apparatus,
wherein advancing the airway tube along the apparatus so that a distal end of the airway tube enters the patient's trachea comprises advancing the airway tube along the second component of the apparatus.

23. Apparatus for introducing an airway tube into a patient's trachea, the apparatus comprising:
an elongated body dimensioned to be received within a lumen of the airway tube, the elongated body having a proximal end, a distal end, and an interior space disposed near the distal end;
an imaging system comprising a pixel array coupled to array driver circuitry, the pixel array disposed within the interior space adjacent to the distal end, the array driver circuitry disposed near the proximal end of the elongated body and electrically coupled to the pixel array and a connector disposed at the proximal end of the elongated body, the connector configured to be coupled to a monitor;
a light source disposed within the interior space adjacent to the imaging system; and
a optical lens associated with the imaging system to focus light rays onto the imaging system.

24. The apparatus of claim 23 further comprising a protrusion extending from the distal end of the elongated body.

25. The apparatus of claim 24 wherein the protrusion is fixed to the distal end of the elongated body.

26. The apparatus of claim 24 wherein the protrusion is movable relative to the distal end of the elongated body.

27. The apparatus of claim 26 wherein the protrusion comprises an elongated member configured to reciprocate through a lumen in the elongated body.

28. The apparatus of claim 26 wherein the protrusion comprises a tension member having a first end coupled to the protrusion and a second end coupled to a handle.

29. The apparatus of claim 28 wherein the protrusion comprises a hollow member that is configured to be at least partially retracted within a space in the elongated body.

30. The apparatus of claim 28 wherein the protrusion is configured to articulate to be retracted within a space in the elongated body.

31. The apparatus of claim 26 wherein the protrusion comprises an inflatable member.

32. The apparatus of claim 23 wherein the elongated body includes a bend near the distal end.

33. The apparatus of claim 23 further comprising a circuit, the circuit having a timer that disables one or more operative features of the apparatus after a pre-determined time.
